(19) United States
(12) Patent Application Publication
(10) Pub. No.: US 2004/0202677 A1
(43) Pub. Date: Oct. 14, 2004

(54) Method of enhanced regional body fat reduction

(76) Inventors: Kevin J. Hopkins, Washington, NJ (US); Gerard Dente, Cedar Grove, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 10/822,248

(22) Filed: Apr. 8, 2004

Related U.S. Application Data
(60) Provisional application No. 60/461,435, filed on Apr. 10, 2003.

(51) Int. Cl. A61K 35/80; A61K 31/555; A61K 31/522

(52) U.S. Cl. 424/195.17; 514/184; 514/263.32

(57) ABSTRACT

A method of enhancing regional body fat reduction is disclosed. The method includes topically applying a fat reduction topical composition on a region of a human body where a regional fat reduction is desired for a treatment period, and administering an amount of a fat reduction oral composition daily during the treatment period. The topical composition includes hydroglycolic fluid extract of Palmaria palmata, Laminaria digitata extract, mannitol, and a pharmaceutically acceptable carrier. The oral composition includes synephrine, methylxanthine, chromium, a herbal combination having diuretic effect and a pharmaceutically acceptable carrier.

### CROSS REFERENCE TO RELATED APPLICATIONS

This application claims the benefit under 35 USC 119 (e) of the provisional patent application Serial No. 60/461,435, filed on Apr. 10, 2003, which is herein incorporated by reference in its entirety.

### FIELD OF THE INVENTION

The present invention relates in general to the field of human body fat reduction, and more particularly to a method of combining a regional body fat reduction with a systemic body fat reduction.

### BACKGROUND OF THE INVENTION

Obesity is defined as an increase in the mass of adipose tissue. Obesity is a problem affecting a vast human population. The weight reduction in obese subjects may be achieved either by medicine or by selected therapeutic diets. However, in both cases, a dramatic weight reduction does not always give satisfactory results from the aesthetic point of view since the fat deposits in some body areas (particularly thighs, and gluteus hips) remain unchanged, causing unpleasant physical disproportion with consequent psychological distress of the patient.

Adipose tissue “fat” is formed by aggregations of fat cells (adipocytes) containing stored fat (lipid) in the form of single droplets of triacylglycerol (triglyceride). Fat tissue is comprised of clusters of adipocytes ranging from small fat cells to large mature fat cells. A single fat cell is 95% fat by volume. The cell nucleus is displaced to one side by the accumulated lipid and the cytoplasm is reduced to a thin rim. Each individual fat cell has large numbers of hormone and other receptors in the cell wall.

Adipose tissue is distributed in the subcutaneous tissue but exhibits regional differences influenced by genes, age, sex, activity levels and eating habits. Infants and young children have a continuous subcutaneous layer of fat. As the young child grows to an adult, the fat layer thins out in some regions of the body but persists and grows thicker in certain sites of predilection. In the male, the principal areas of predilection are the neck and the region overlying the seventh cervical vertebra, the subcutaneous area overlying the deltoid and triceps, the lumbosacral region, and the buttocks. In the female, subcutaneous fat is most abundant in the anterior neck, the breasts, the buttocks, the epitrochanteric region, and the anterior aspect of the thigh.

The aesthetic problem for most individuals who achieve modest or even significant degrees of weight loss, is that the adipose tissue volume reduction is often not lost from the specific anatomical sites they desire (e.g. tummy, buttock, thigh), but occurs rather unpredictably from all anatomical areas.

One of the major contributors to body weight homeostasis in the human body is the adrenergic system. There are two types of adrenergic receptors, alpha and beta, as well as subtypes of each, and depending on which are activated, lipolysis (breakdown of fat) can be either stimulated or inhibited. The most well-known adrenoreceptors are the beta receptors (there can be divided into subtypes 1, 2, and 3). It is through these receptors that drugs such as the ephedrine/caffeine stack and Clenbuterol exert their effects.

To achieve a regional fat reduction, various topical fat loss product have been developed in the past two decades. Codif Recherche et Developpement, France, developed a topical gel containing a hydroglycolic fluid extract of Palmaria palmta (a red seaweed of the Rhodophyceae class), marketed under the trade name Rhodofiltrat®. The test in human subjects showed that Rhodofiltrat® increases cutaneous microcirculation. It is believed that Rhodofiltrate acts on capillary blood vessel and activates peripheral microcirculation with increase in cell exchange and cell matter elimination desired for slimming treatment. It functions as a vasodilating agent.

Codif Recherche et Developpement, France, also developed a topical gel with a combination of Rhodofiltrat® and a Laminaria digitata extract which is marketed under the trade name Phyco® R75. The laboratory results have demonstrated that Phyco® R75 increases cAMP level (cyclic monophosphate adenosyl) in human adipocytes. It was found that 1% of Phyco® R75 has the similar effect in stimulating cAMP production in human adipocytes as 10⁻⁴ M of caffeine. The increase of the cAMP intracellular concentration stimulates the lipase which is responsible for hydrolysis of triglycerides. It is believed that the increase of cAMP by Phyco® R75 is achieved by its function of inhibiting binding of neuropeptide to its receptor and inhibiting binding of adrenalin to alpha2 receptor and inhibiting phosphodiesterase. Phyco® R75 has been used as an effective slimming ingredient in cosmetic products because of its lipolysis function. It is further believed that to combine Phyco® R75 with Rhodofiltrat® can make the drainage easier and avoid stasis and plasmic exudation.

Mannitol is a sweetener, stabilizer, humectant and bulking agent in foods and supplements. Mannitol is found in abundance in nature, particularly in exudates from trees, and in marine algae and fresh mushrooms. It has a molecular formula of C₆H₈(OH)₆, an isomer of sorbitol. Mannitol is typically produced today by the hydrogenation of specialty glucose syrups. It is commercially available in variety of powder and granular forms.

Mannitol has also been used medically. It is known that when administered systemically, mannitol increases the osmolarity of the glomerular filtrate, which decreases the reabsorption of water and increases excretion of sodium and chloride. It also increases the osmolarity of the plasma, which causes enhanced flow of water from tissues into the interstitial fluid and plasma. It is diuretic and is used to prevent or treat the oliguric phase of acute renal failure before irreversible renal failure occurs. It is also used to decrease ICP and cerebral edema by decreasing brain mass, and to decrease elevated intraocular pressure when the pressure cannot be lowered by other means. Mannitol is dramatically effective in reversing acute brain swelling. It is also used to promote urinary excretion of toxic substances, and as a urinary irrigant to prevent hemolysis and hemoglobin buildup during transurethral prostatic resection or other transurethral surgical procedures.

On the other hand, various improved or new chemicals, particularly extracts of natural products, have been developed recently for systemic body weight loss and control. U.S. Pat. No. 5,804,596 teaches the use of forskolin, a diterpenoid compound extracted from Coleus forskohlii, for promoting lean body. Forskolin activates adenylate cyclase, the enzyme involved in the production of cAMP which stimulates the lipase responsible for hydrolysis of triglycerides. Moreover, among those known chemicals, caffeine is effective in stimulating cAMP production in human adipocytes.

U.S. Pat. Nos. 5,087,623, 5,087,624 and 5,175,156 teach that chromium picolinate has an anabolic effect, which can be orally administered to increase lean body mass.

Theobromine is a methylxanthine, in the same class of compounds as caffeine. Theobromine, 3,7-dihydro-3,7-dimethyl-1H-purine-2,6-dione, is the primary methylxanthine found in products of the cocoa tree, theobroma cacao. Cocoa beans naturally contain approximately 300-1200 mg/ounce theobromine. All chocolate products contain theobromine. Theobromine affects humans similarly to caffeine, but on a much smaller scale.

Theobromine is mildly diuretic and a mild stimulant. Theobromine has been used as a drug for its diuretic effect, particularly in cases where cardiac failure has resulted in an accumulation of body fluid. Because of its ability to dilate blood vessels, theobromine also has been used to treat high blood pressure.

Although various regional fat reduction products and oral products have been developed, most time they are used separately without complimentary support from each other. The overall effects of weigh loss and body shape control are not optimal. Therefore, it is apparent that there exists a need for an improved systemic, and synergetic approach for enhancing effect of body fat reduction, particularly regional fat reduction.

### SUMMARY OF THE INVENTION

The present invention provides a method of enhancing regional body fat reduction. The method comprises the steps of topically applying a fat reduction topical composition daily on a region of a human body where a regional fat reduction is desired for a treatment period, the topical composition consisting essentially of a topical vasodilator, an adrenergic agent enabling increasing of cAMP, a diuretics and a pharmaceutically acceptable carrier; and administering an effective amount of a fat reduction oral composition daily during the treatment period, the oral composition comprising an alpha adrenergic agent, a chromium source material, methylxanthine, a herb exerting diuretic effect, and a pharmaceutically acceptable carrier.

In a preferred embodiment, the topical composition contains mannitol as a the diuretics, laminaria digitata extract as the adrenergic agent; and the topical vasodilator is one selected from the group consisting of hydroglycolic fluid extract of Palmaria palmate, Ergoloid mesylates, papaverine, isoxsuprine HCI, ethaverine HCI, isosorbide mono- and di-nitrates, nitroglycerine, and combination thereof. The oral composition contains synephrine as the alpha adrenergic agent; caffeine, theobromine, and a herbal combination consisting of Couchgrass rhizome, Buchu leaf, Uva ursi leaf, Juniper erry, Hydrangea root and Cornsilk stylus.

### DETAILED DESCRIPTION OF THE INVENTION

In one embodiment, the present invention provides a method of enhancing regional body fat reduction. The method comprises the steps of: (a) topically applying a fat reduction topical composition on a region of a human body where a regional fat reduction is desired for a treatment period; the topical composition comprising a topical vasodilator, such as hydroglycolic fluid extract of Palmaria palmate and other topical vasodilators; an adrenergic agent enabling increase of cAMP, such as Laminaria digitata extract or other adrenergic agents; a diuretics, such as mannitol or other agents having diuretic effect; and a pharmaceutically acceptable carrier; and (b) administering orally an amount of a fat reduction oral composition daily during the treatment period; the oral composition comprising methylxanthine, such as caffeine, theobromine; an alpha adrenergic agent, such as synephrine; a chromium source material such as chromium picolinate; an herbal combination exerting diuretic effect; and a pharmaceutically acceptable carrier.

Preferably, the active components of the topical composition are hydroglycolic fluid extract of Palmaria palmata, Laminaria digitata extract and mannitol. The hydroglycolic fluid extract of Palmaria palmata is commercially available under the trade name Rhodofiltrat® from Codif, 70 rue du Commandant I′Herminier, B. P. 40-35404 Saint-Malo cedex, France. Other suitable examples of vasolilator include Ergoloid mesylates, papaverine, isoxsuprine HCI, ethaverine HCI, isosorbide mono- and di-nitrates, and nitroglycerine. Laminaria digitata extract is also commercially available under the trade name Phyco® R-75 from Codif. Mannitol is commercially available in variety of powder and granular forms.

The topical composition preferably further comprises an antimicrobial as preservative. Suitable antimicrobials for the topical composition include, but not limited to, diazolidinyl urea, methylparaben, propylparaben, disodium and tetrasodium EDTA. The pharmaceutically acceptable carriers in the topical composition include, but not limited to, water, hydroxyethylcellulose, polysorbate 20, and other suitable carriers.

Optionally, the topical composition can further comprise a buffer for adjusting and maintaining pH of the composition. Suitable examples include an acid, such as citric acid, and a base, such as sodium hydroxide. Furthermore, the topical composition can also comprise a pharmaceutically acceptable dye for providing a desirable color to the topical composition. Suitable examples of the dyes include FD&C Bule No. 1, FD&C Red No. 40, FD&C Yellow No. 5. Moreover, the topical composition can further comprise a fragrance. Suitable examples of fragrance include lemon oil, orange oil, grapefruit oil, palmarosa oil, and jasmine oil.

In one embodiment, the topical composition comprises hydroglycolic fluid extract of palmaria palmata in a concentration range from about 2% to 10%, laminaria digitata extract in a concentration range from about 0.2% to 5%, and mannitol in a concentration range from about 0.2% to 2%. In one example, the topical composition comprises 5% (w/w) Rhodofiltrat®, 1% (w/w) Phyco® R75, and 0.5% (w/w) mannitol. The composition also includes carriers, hydroxyethylcellulose and polysorbate 20; fragrance oil; antimicrobials, diazolidinyl urea, methylparaben, propylparaben, and tetrasodium EDTA; and citric acid for adjusting pH.

Each of the three active components of the topical composition has its individual functionality, and more importantly, their combined functionality. For example, at an individual level, Phyco® R75 increases cAMP level in human adipocytes, which promotes the lipolysis process. Rhodofiltrat® activates peripheral microcirculation with increase in cell exchange and cell matter elimination desired for the regional fat reduction. Mannitol reduces cellular water retention. The combination of Phyco® R75, a lipolysis promoter; Rhodofiltrat®, a vasodilating agent; and mannitol, a cellular flux enhancer, provides a strong synergetic effect to activate a local lipolysis and enhance removal of the hydrolysis product of triglycerides in the region where the topical composition is applied.

In the other aspect of the present invention, the oral composition preferably to be in a form of capsule or tablet. Other pharmaceutical dosage forms, such as pill, gel and liquid, can also be used. The oral composition comprises from about 50 μg to about 500 μg of chromium picolinate, from about 1 mg to about 500 mg of caffeine, and from about 1 mg to about 1,000 mg of theobromine per capsule. The oral composition can further include a herbal combination which has diuretic effect. A suitable example of the herbal blend includes Couchgrass rhizome, Buchu leaf, Uva ursi leaf, Juniper erry, Hydrangea root, and Cornsilk stylus. Preferably, the daily dosage of the oral composition is to administer from about 50 μg to about 400 μg of chromium picolinate, from about 50 mg to 300 mg of caffeine, and from about 140 mg to about 840 mg of Theobroma cacau extract (about 12% of theobromine). Preferably, the caffeine is extracted from guarana seed and green tea leaf. Theobroma cacau extract can be obtained commercially from FCC Products, Gerry Woods, N.J. Another example of the oral composition is shown in Example 1 hereinafter. Chromium picolinate is commercially available.

The pharmaceutically acceptable carriers in the oral composition include, but not limited to, gelatin, cellulose, dicalcium phosphate, magnesium stearate and silica.

The combination of chromium picolinate caffeine or other xanthine derivatives; synephrine or other adrenergic amine, also provides a synergetic effect for promoting lipolysis and removal of hydrolysis product of the body fat at a system level.

During the enhanced regional body fat reduction treatment, it is preferred to administer one to two capsules of the oral composition one to two times daily. The topical composition can be applied one or twice daily at the body areas where the fat reduction is desired during the treatment period. Preferably, the treatment period is four weeks and longer.

With the combination of the topical composition and oral composition of the present invention, the regional body fat reduction can be further supported and enhanced by the systemic fat reduction effect promoted by the oral composition, particularly enhanced systemic removal of regionally generated triglycerides hydrolysis product. Using the method of enhancing regional body fat reduction of the present invention, one can achieve an overall balanced body fat reduction with desirable aesthetic effects.

### EXAMPLE 1

An oral composition in capsule form contains following amount of components per capsule:

| | |
|---|---|
| Citrus aurantium extract (6% synephrine) | 125 mg |
| (immature fruit) | |
| Caffeine (from green teas leaf extract | 50 mg |
| and guarana seed) | |
| Theobrama cacau extract (12% theobromine) | 140 mg |
| Chromium (chromium picolinate) | 50 μg |
| Herbal blend: | 100 mg |
| Couchgrass rhizome, Buchu leaf, Uva ursi leaf, | |
| Juniper erry, Hydrangea root, Cornsilk stylus | |

The oral composition further comprises inert carriers including dicalcium phosphate, microcrystalline cellulose, croscarmellose sodium, stearic acid, magnesium stearate, and silica.

A topical composition in a gel form contains following amount of components:

| | |
|---|---|
| Rhodofiltrat depalmaria palm | 5.0% |
| Phyco R 75 (Laminaria digitata extract) | 1.0% |
| Mannitol | 0.5% |
| Denatured alcohol 40-2 | 10.0% |
| Natrosol 250 HHR (hydroxyethycellulose) | 1.4% |
| Tween-20 ® (polysorbate 20) | 0.5% |
| Versene 100XL (Na₄EDTA) | 0.1% |
| Antimicrobials | 0.5% |
| Citric acid | 0.01% |
| Water | 80.95% |

Wherein the denatured alcohol is used as an astringent. Natrosol 250 HHR is a product of Hercules Incorporated, Aqualon Division, Wilmington, Del. Versene 100 XL is a product of Dow Chemical Company, Midland, ^{Mich.}

### EXAMPLE 2

A twenty one year old male were in an excise program for three months prior to using the oral and topical compositions of Example 1. The excise program included working out three times per week with weight training and the treadmill exercise for thirty minutes. The subject used the body fat reduction treatment method of the present invention for two months, which included orally administrating two tablets of the oral composition of Example 1 twice daily and topically applying the topical composition of Example 1 twice daily. During the two month treatment, the subject maintained a regular diet. The following parameters were measured prior to and two month after using the oral and topical composition of Example 1.

| Parameters | Before | Two Month After |
|---|---|---|
| Body weight | 214 lb. | 202 lb. |
| Body fat percentage | 20.4% | 16.2% |
| Waist Measurement | 39″ | 37″ |

### EXAMPLE 3

A twenty six year old female with a history of working out with weights and cardiovascular exercise was treated with the body fat reduction treatment method of the present invention for two months. The treatment included orally administrating two tablets of the oral composition of Example 1 twice daily and topically applying the topical composition of Example 1 twice daily. During the treatment, her diet and exercise program remained the same. The following parameters were measured prior to and two months after the treatment.

| Parameters | Before | Two Month After |
|---|---|---|
| Body weight | 131 lb. | 124 lb. |
| Body fat percentage | 18.6% | 15.7% |

### EXAMPLE 4

A male bodybuilder with an extensive background in weight training was treated with the instant body fat reduction treatment method using the oral and topical compositions of Example 1, in conjunction with a decreased carbohydrate diet. The subject orally administrated two tablets of the oral composition of Example 1 twice daily and topically applying the topical composition of Example 1 twice daily. His progress was dramatic as compared to the previous pre-contest preparatory diets. In only one month, the following results were observed.

| Parameters | Before | One Month After |
|---|---|---|
| Body weight | 191 lb. | 182 lb. |
| Body fat percentage | 8.2% | 5.0% |
| Skinfold thickness* on waist | 8.0 mm | 5.0 mm |
| Skinfold thickness on chest | 6.9 mm | 4.0 mm |
| Skinfold thickness on thigh | 10.0 mm | 6.5 mm |

*Skinfold thickness is measured using the same skinfold callipers during the treatment period.

While the present invention has been described in detail and pictorially shown in the accompanying drawings, these should not be construed as limitations on the scope of the present invention, but rather as an exemplification of preferred embodiments thereof. It will be apparent, however, that various modifications and changes can be made within the spirit and the scope of this invention as described in the above specification and defined in the appended claims and their legal equivalents.

What is claimed is:

1. A method of enhancing regional body fat reduction comprising the steps of:
(a) topically applying a fat reduction topical composition daily on a region of a human body where a regional fat reduction is desired for a treatment period, said topical composition consisting essentially of hydroglycolic fluid extract of Palmaria palmata, Laminaria digitata extract, mannitol and a pharmaceutically acceptable carrier; and
(b) administering an effective amount of a fat reduction oral composition daily during the treatment period, said oral composition comprising synephrine, methylxanthine, chromium source material, and a pharmaceutically acceptable carrier.

2. The method of enhancing regional body fat reduction of claim 1, wherein said treatment period is four weeks or more.

3. The method of enhancing regional body fat reduction of claim 2, wherein said methylxanthine is one selected from the group consisting of caffeine, theobromine, and combination thereof.

4. The method of enhancing regional body fat reduction of claim 3, wherein said chromium source material is chromium picolinate.

5. The method of enhancing regional body fat reduction of claim 4, wherein a daily dosage of said oral composition is from about 50 μg to about 400 μg of chromium picolinate, from about 50 mg to 300 mg of caffeine, and from about 17 mg to about 100 mg of theobromine.

6. The method of enhancing regional body fat reduction of claim 5, wherein said oral composition further comprises a herb having diuretic effect.

7. The method of enhancing regional body fat reduction of claim 6, wherein said herb is one selected from the group consisting of Couchgrass rhizome, Buchu leaf, Uva ursi leaf, Juniper erry, Hydrangea root, Cornsilk stylus, and combination thereof.

8. The method of enhancing regional body fat reduction of claim 2, wherein said topical composition consisting essentially of said hydroglycolic fluid extract of palmaria palmata in a concentration range from about 2% to 10%, said laminaria digitata extract in a concentration range from about 0.5% to 5%, and said mannitol in a concentration range from about 0.2% to 2%.

9. A method of enhancing regional body fat reduction comprising the steps of:
(a) topically applying a fat reduction topical composition daily on a region of a human body where a regional fat reduction is desired for a treatment period, said topical composition consisting essentially of hydroglycolic fluid extract of Palmaria palmata, Laminaria digitata extract, mannitol and a pharmaceutically acceptable carrier; and
(b) administering an effective amount of a fat reduction oral composition daily during the treatment period, said oral composition comprising synephrine, caffeine, theobromine, chromium picolinate, a herbal combination having diuretic effect and a pharmaceutically acceptable carrier.

10. The method of enhancing regional body fat reduction of claim 9, wherein said treatment period is four weeks or more.

11. The method of enhancing regional body fat reduction of claim 10, wherein said topical composition consisting essentially of said hydroglycolic fluid extract of palmaria palmata in a concentration range from about 2% to 10%, said laminaria digitata extract in a concentration range from about 0.5% to 5%, and said mannitol in a concentration range from about 0.2% to 2%.

12. The method of enhancing regional body fat reduction of claim 11, wherein said topical composition consisting essentially of about 5% of said hydroglycolic fluid extract of palmaria palmata, about 1% of said laminaria digitata extract, and about 0.5% of said mannitol.

13. The method of enhancing regional body fat reduction of claim 9, wherein a daily dosage of said oral composition is from about 50 μg to about 400 μg of chromium picolinate, from about 50 mg to 300 mg of caffeine, and from about 17 mg to about 100 mg of theobromine.

14. The method of enhancing regional body fat reduction of claim 13, wherein said herbal combination comprises Couchgrass rhizome, Buchu leaf, Uva ursi leaf, Juniper erry, Hydrangea root, Cornsilk stylus.

15. A method of enhancing regional body fat reduction comprising the steps of:
(a) topically applying a fat reduction topical composition daily on a region of a human body where a regional fat reduction is desired for a treatment period, said topical composition consisting essentially of a topical vasodilator, an adrenergic agent enabling increasing of cAMP, a diuretics and a pharmaceutically acceptable carrier; and
(b) administering an effective amount of a fat reduction oral composition daily during the treatment period, said oral composition comprising an alpha adrenergic agent, a chromium source material, methylxanthine, a herb exerting diuretic effect, and a pharmaceutically acceptable carrier.

16. The method of enhancing regional body fat reduction of claim 15, wherein said diuretics in said topical composition is mannitol.

17. The method of enhancing regional body fat reduction of claim 16, wherein said adrenergic agent in said topical composition is a laminaria digitata extract.

18. The method of enhancing regional body fat reduction of claim 17, wherein said topical vasodilator in said topical composition is one selected from the group consisting of hydroglycolic fluid extract of Palmaria palmate, Ergoloid mesylates, papaverine, isoxsuprine HCI, ethaverine HCI, isosorbide mono- and di-nitrates, nitroglycerine, and combination thereof.

19. The method of enhancing regional body fat reduction of claim 15, wherein said alpha adrenergic agent in said oral composition is synephrine.

20. The method of enhancing regional body fat reduction of claim 15, wherein said herb is one selected from the group consisting of Couchgrass rhizome, Buchu leaf, Uva ursi leaf, Juniper erry, Hydrangea root, Cornsilk stylus, and combination thereof.
